SEQUENCE LISTING (10 sequences)

SEQ ID NO: 1 - DNA, 20 bp, Artificial
  PCR Primer
ccatctcatccctgcgtgtc

SEQ ID NO: 2 - DNA, 20 bp, artificial
  PCR Primer
cctatcccctgtgtgccttg

SEQ ID NO: 3 - DNA, 28 bp, artificial
  UT PCR Primer
ggcggcgaccatctcatccctgcgtgtc

SEQ ID NO: 4 - DNA, 19 bp, artificial
  PCR Primer
gcctccctcgcgccatcag

SEQ ID NO: 5 - DNA, 19 bp, artificial
  PCR Primer
gccttgccagcccgctcag

SEQ ID NO: 6 - DNA, 27 bp, artificial
  UT PCR Primer
ggcggcgagcctccctcgcgccatcag

SEQ ID NO: 7 - DNA, 19 bp, artificial
  PRC Primer
acactctttccctacacga

SEQ ID NO: 8 - DNA, 18 bp, artificial
  PCR Primer
caagcagaagacggcata

SEQ ID NO: 9 - DNA, 27 bp, artificial
  UT PCR Primer
ggcggcgaacactctttccctacacga

SEQ ID NO: 10 - DNA, 8 bp, artificial
  Universal Probe Sequence
ccgccgct

(19) United States
(12) Patent Application Publication
(10) Pub. No.: US 2010/0069250 A1
(43) Pub. Date: Mar. 18, 2010

(54) Digital PCR Calibration for High Throughput Sequencing

(75) Inventors: Richard Allen White, III, Hayward, CA (US); Stephen R. Quake, Stanford, CA (US); Hei-Mun Christina Fan, Mountain View, CA (US); Paul Blainey, Mountain View, CA (US)

Correspondence Address: PETERS VERNY , L.L.P., 425 SHERMAN AVENUE, SUITE 230, PALO ALTO, CA, 94306, US

(73) Assignee: The Board of Trustees of the Leland Stanford Junior University, Palo Alto, CA (US)

(21) Appl. No.: 12/541,722

(22) Filed: Aug. 14, 2009

Related U.S. Application Data
(60) Provisional application No. 61/089,513, filed on Aug. 16, 2008.

(51) Int. Cl. C40B 20/04; C40B 30/08; C12Q 1/68; C07H 21/04

(52) U.S. Cl. 506/4; 506/11; 435/6; 536/24.3; 536/24.33

(57) ABSTRACT

Disclosed is a method for accurately determining the number of template molecules in a library of nucleic acids (e.g., DNA) to be sequenced. The method does not require large amounts of the DNA sample, nor does it require the preparation of a standard curve. The method is especially applicable to methodologies for “sequencing by synthesis,” where quantitation of the starting library is important. The method uses quantitative real time PCR, especially digital PCR, which measures the number of individual molecules in a sample. The present method particularly may use a microfluidic device for running large numbers of PCR reactions. Each PCR reaction is monitored in real time by a primer/probe combination. The forward primer is adapted to contain a sequence not on the adapter but which corresponds to a probe sequence. A short probe which generates fluorescence during the PCR process is used.

### CROSS-REFERENCE TO RELATED APPLICATIONS

This application claims priority from U.S. Provisional Patent Application No. 61/089,513, filed on Aug. 16, 2008, which is hereby incorporated by reference in its entirety.

### STATEMENT OF GOVERNMENTAL SUPPORT

This invention was made with U.S. Government support under contract OD000251 awarded by the National Institutes of Health. The Government has certain rights in this invention.

### REFERENCE TO SEQUENCE LISTING, COMPUTER PROGRAM, OR COMPACT DISK

Applicants assert that the paper copy of the Sequence Listing is identical to the Sequence Listing in computer readable form found on the accompanying computer file. Applicants incorporate the contents of the sequence listing by reference in its entirety.

### BACKGROUND OF THE INVENTION

1. Field of the Invention

The present invention relates to the field of nucleic acid measurement, and, in particular to DNA quantitation.

2. Related Art

Presented below is background information on certain aspects of the present invention as they may relate to technical features referred to in the detailed description, but not necessarily described in detail. That is, certain components of the present invention may be described in greater detail in the materials discussed below. The discussion below should not be construed as an admission as to the relevance of the information to the claimed invention or the prior art effect of the material described.

A new generation of sequencing technologies are revolutionizing biology, biotechnology, and medicine. These technologies are based on “sequencing by synthesis” and have been commercially deployed in significant numbers. They are also known as “massively parallel sequencing,” (which may or may not involve sequencing by synthesis.) A key advance facilitating higher throughput and lower costs for several of these platforms was migration from clone-based sample preparation commonly used in Sanger sequencing to massively parallel clonal PCR amplification of sample molecules on beads, as exemplified by the products of 454 Life Sciences, Branford, Conn., or amplification of sample molecules on a surface by bridge PCR, as exemplified by the products of Solexa, Inc., Hayward, Calif. (now part of Illumina, Inc.). The Solexa process, as described in BioTechniques® Protocol Guide 2007, Published December 2006: p 29, utilizes single molecule clonal amplification which involves six steps: template hybridization, template amplification, linearization, blocking 3′ ends, denaturation and primer hybridization. In contrast to the 454 and ABI methods which use a bead-based emulsion PCR to generate “polonies”, the Solexa method utilizes a unique “bridged” amplification reaction that occurs on the surface of the flow cell.

In the known sequencing by synthesis methods, the parallel amplification steps are relatively efficient, with sequence data obtained from a significant fraction of sequencing library molecules input to the amplification. Thus the term “library” is used in its art-recognized sense, that is a collection of nucleic acid molecules (RNA, cDNA or genomic DNA) obtained from a particular source being studied, such as a certain differentiated cell, or a cell representing a certain species (e.g., human). The library may be processed according to requirements of the study undertaken, and will be further processed according to the needs of the sequencing protocol to be used. As discussed below, the sequencing protocols involve adapters ligated to the ends of the molecules to be sequenced. The adapters are typically 10-20 bp; fragments of DNA to be sequenced are 150-300 by in length; and RNA fragments will typically be smaller. A massively parallel sequencing method, when coupled with a good loading efficiency onto the instrument, results in on the order of one million library molecules (typically less than a picogram of library DNA) being required to carry out a full sequence run. However, these processes, as recommended by the manufacturers, require one to ten trillion (typically 1-5 micrograms) DNA fragments as input for library preparation. This is primarily because quantitation of the library DNA according to the manufacturers' protocols consumes more than a billion molecules, and secondarily because of the limited efficiency of the library preparation methods, which have typical conversion efficiencies of 0.01%-1%.

The requirement for micrograms of input DNA limits the pool of samples that next generation sequencing technologies have the ability to sequence, since for many applications microgram quantities of sample are not available. In some cases it is possible to use amplification such as PCR or MDA (multiple displacement amplification). MDA is further described in Hellani et al., “Multiple displacement amplification on single cell and possible PGD,” Mol. Hum. Reprod. Advance Access originally published online on Oct. 1, 2004 Molecular Human Reproduction 2004 10(11):847-852.

However, amplification of samples may have bias and introduce distortion. The present method, described below, on the other hand, provides a method for highly accurate absolute quantitation of sequencing libraries that consumes subfemptogram amounts of library material. Eliminating the large quantity requirement for traditional quantitation has the direct effect of reducing the sample input requirement from trillions of fragments (micrograms) to billions of fragments (nanograms) or less, opening the way for minute and/or precious samples onto the next-generation sequencing platforms without the distorting effects of pre-amplification steps.

The standard workflow for the next-generation instruments using sequencing by synthesis entails library creation, (requiring a bulk PCR step on the “bridged” amplification reaction), massively parallel PCR amplification of library molecules, followed by sequencing. Library creation starts with conversion of the sample to appropriately sized fragments, ligation of adaptor sequences onto the ends of the sample molecules, and selection for molecules properly appended with adaptors. The presence of the adaptor sequences on the ends of the library molecules enables amplification of random-sequence inserts by PCR. The number of library DNA molecules in the massively parallel PCR step is critical: it must be low enough that the chance of two DNA molecules associating with the same bead in emulsion PCR (Roche/454) or the same surface patch in bridge PCR (Illumina/Solexa) is low, but there must be enough library DNA present such that the yield of amplified sequences is sufficient to realize a high sequencing throughput.

The standard workflow from manufacturers of high throughput sequencing manufacturers typically calls for measuring the mass of library DNA using the Agilent 2100 Bioanalyzer capillary gel electrophoresis (GE) instrument (454 Life Sciences), which is used to try to quantify the library electrophoretically, or the nanodrop spectrophotometer (Nanodrop Technologies at nanodrop.com), and then converting the mass to a number count by using knowledge of the length distribution.

Table 1 below compares current sequencing library quantitation methods with the present method (last two columns):

TABLE 1
Comparison of Sequencing Library Quantitation Methods

| | | | | | | UT- |
|---|---|---|---|---|---|---|
| | | Capillary | | Real-time | UT- | digital |
| Method: | Nanodrop | GE | Ribogreen | PCR | qPCR | QPCR |
| Detection | UV absorption | Intercalating | Intercalating | Syber Green I | Hydrolysis | Hydrolysis |
| Chemistry: | Thermo | fluorophore | fluorophore | Intercalating | probe | probe |
| Companies: | Scientific | Aligent, Bio- | Invitrogen | fluorophore | (Taqman) | (Taqman) |
| | | Rad | | Many | Many | Fluidigm |
| LOQ:* | 2 ng** | 25 ng | 1 ng | (0.3 fg) | (0.03 fg) | (0.03 fg) |
| | (7.2 billion | (91 billion | (3.6 billion | 1000 copies | 100 copies | 100 copies |
| | copies) | copies) | copies) | | | |
| Quantitation | Mass/ | Mass/ | Mass/ | Mass/ | Molecules/ | Molecules/ |
| Modality: | Absolute | Relative | relative | relative | relative | absolute |
| Quantitation | No standard | Required - | Required - | Required - | Required - | No |
| Standard: | necessary | calibrated by | calibrated | calibrated by | calibrated | standard |
| | | mass | by mass | mass | by mass | necessary |
| Reference | nanodrop.com | Ricicova | Jones | Simpson | Zhang | Vogelstein |
| | | (2003) | (1998) | (2000); | (2003); | (1999); |
| | | | | Meyer | present | present |
| | | | | (2008) | method | method |

In Table 1, LOQ indicates the limit of quantitation for an ssDNA 500-mer. The asterisk (*) indicates a value which the manufacturer does not specify as LOD or LOQ. LOQ is the true limit of the quantification of an instrument or biochemical assay, as this is a practical quantitation limit that detects the true material over what is still noise. LOD measurement could be detecting noise or a blank sample. The present method requires no standard, and, because of the use of real time quantitative PCR and digital analysis, counts nondegraded molecules rather than mass.

Quantification of the library by mass presents three major stumbling blocks that effectively render the quantification inaccurate to the degree where the sequencing results can be adversely affected. First, mass-based quantitation also requires an accurate estimate of the length of the molecules to determine the molar concentration of DNA fragments. Second, degraded and damaged molecules that cannot be amplified in the massively parallel amplification step are counted. And third, methods of measuring DNA mass lack sensitivity, and are imprecise in concentration measurements near the limit of detection.

When the library concentration is underestimated, the possibility of molecular crosstalk arises where the clonality of beads (454) or clusters (“bridged” amplification reaction) is compromised, reducing the fraction of useful reads. When the library concentration is overestimated, the number of beads recovered (454) or number of clusters generated (“bridged” amplification reaction) is reduced, in which case the full capacity of the sequencers cannot be used. Before carrying out a bulk sequencing run with a new library, Roche and Illumina recommend carrying out a four-point titration run on their sequencers in order to empirically determine the optimal volume of DNA for the massively parallel PCR. Illumina's Solexa sequencing preparation strictly depends on the accuracy of library quantitation. Illumina's platform does have the user quality check the library with traditional Sanger Sequencing before use. The digital PCR method disclosed below eliminates all three of these problems and the requirement for titration.

Recently, Meyer et al. (ref. 5) developed a SYBR® Green real-time PCR assay that allows the user to estimate the number of amplifiable molecules in sequencing trace samples. (Note: SYBR is a registered trademark of Molecular Probes, Inc. and the dye may be covered by U.S. Pat. No. 5,436,134.) This was the first report of PCR-based quantitation of sequencing libraries, and extended the sensitivity of library quantitation significantly, although to an essentially unknown extent, since the source material used to make the trace libraries was not quantitated. However, the SYBR Green assay presents two principle disadvantages: 1) SYBR Green I dye is an intercalating fluorochrome that gives signal in proportion to DNA mass, not molecule number, 2) the SYBR Green assay relies on a external standard that limits the absolute accuracy over time and is not universal to all sample types. The standard must have the same amplification efficiency and molecular weight distribution as the unknown library sample. This means that the user must have on hand a bulk sequencing library very similar to the trace library being made and that the molecular weight distributions of both the standard and the new library be known—often impractical for a trace sample library. Furthermore, this standard library must be of extremely high quality if mass-based quantitation is to be used to calibrate the assay for amplifiable molecules, which makes assessment of the concentration of amplifiable molecules in a degraded sample extremely difficult. Lastly, sequence-nonspecific detection chemistries like SYBR Green give signal from all dsDNA products generated, including primer dimers and nonspecific amplification products, which may be an issue in complex samples.

In particular, side products can compete with specific amplification from low numbers (<1000) of template molecules, limiting the accuracy of SYBR Green quantitation for dilute samples (Simpson 2000). Although the presence of these side products can often be discerned by analysis of the product melting curve, opportunities to optimize the primers are limited due to the short length of the adaptor sequences and the specific nucleotide sequences required for compatibility with proprietary sequencing reagents. Sensitivity to side products gives SYBR Green a tendency toward overestimation of the sample quantity. The present invention, as described below, comprises an assay that circumvents limitations of using TaqMan® detection chemistry and the digital PCR modality. TaqMan® is a registered trademark of Roche Molecular Systems, Inc.

TaqMan® detection chemistry has the advantage of yielding a fluorescence signal proportional to the number of molecules that have been amplified, not by the total mass of dsDNA in the sample. The method is more fully described in Heid et al., “Real Time Quantitative PCR,” Genome Research, 6:986-995 (1996). This method, herein termed “real time PCR,” works by the addition of a double-labeled oligonucleotide probe in a PCR reaction powered by the 5′ to 3′ exonuclease activity of the polymerase. However, the probe must be complimentary to one of the two product strands such that the extending polymerase will encounter it and separate the two labels by exonuclease activity, activating the probe's fluorescence. Conventional TaqMan® detection chemistry requires that the probe is complementary to the region within the amplified portion of the template between the two amplification primers. This strategy fails for the sequencing libraries, which have inserts of unknown or random sequence between short adaptor sequences.

Currently four different chemistries, (1) TaqMan® (Applied Biosystems, Foster City, Calif., USA), (2) Molecular Beacon probes, (available from Biosearch Technologies), (3) Scorpion® probes (available from Sigma-Aldrich) and (4) SYBR® Green (Molecular Probes), are available for real-time PCR. TaqMan probes are hydrolysis probes developed by Applied Biosystems to increase the specificity of real-time PCR assays. The TaqMan probe principle relies on the 5′-3′ nuclease activity of Taq polymerase to cleave a dual-labeled probe during hybridization to the complementary target sequence and fluorophore-based detection. Molecular Beacon probes, developed at the Public Health Research Institute of New York, are further described in U.S. Pat. Nos. 5,210,015; 5,487,972; 5,804,375; and 5,994,076. Molecular Beacon Probes are DNA oligonucleotides that become fluorescent when they hybridize to their target. They are hairpin-shaped, single-stranded molecules consisting of a probe sequence embedded between complementary sequences that form a hairpin stem. Scorpions® is a registered trademark of DxS Ltd. Scorpion probes are further described in US 2005/0164219 by Whitcombe, et al., published Jul. 28, 2005, entitled “Methods and primers for detecting target nucleic acid sequences.” The Scorpion primer carries a Scorpion probe element at the 5′ end. The probe is a self-complementary stem sequence with a fluorophore at one end and a quencher at the other. The Scorpion primer sequence is modified at the 5′ end. It contains a PCR blocker at the start of the hairpin loop, and HEG monomers are typically added as blocking agents.

Chemistries which allow detection of PCR products via the generation of a fluorescent signal can be adapted, given the teachings below, to the present method. TaqMan probes, Molecular Beacons and Scorpions depend on Förster Resonance Energy Transfer (FRET) to generate the fluorescence signal via the coupling of a fluorogenic dye molecule and a quencher moiety to the same or different oligonucleotide substrates, and are potentially useful in the present methods. On the other hand, SYBR Green is a fluorogenic dye that exhibits little fluorescence when in solution, but emits a strong fluorescent signal upon binding to double-stranded DNA.

### Specific Patents and Publications

Zhang et al., “A novel real-time quantitative PCR method using attached universal template probe,” Nuc. Acid. Res., 31, page e123 (8 pp) discloses the use of a universal template (UT) probe which is an approximately 20 base attachment to the 5′ end of a PCR primer and can hybridize to a complementary Taqman probe.

Kambara et al., “DNA sequencing method and DNA sample preparation method,” U.S. Pat. No. 5,985,556, issued Nov. 16, 1999, discloses a method of DNA sequencing including digesting a sample DNA with a restriction enzyme to obtain a DNA fragment; introducing an oligonucleotide having a definite base sequence into the DNA fragment at the 3′ terminus; and performing a complementary strand extension reaction, using a labeled primer.

Adessi et al., “Methods of nucleic acid amplification and sequencing,” U.S. Pat. No. 7,115,400, issued Oct. 3, 2006, listing as assignee Solexa Ltd., discloses methods of nucleic acid amplification and sequencing, and describes new methods of solid-phase nucleic acid amplification which enable a large number of distinct nucleic acid sequences to be arrayed and amplified simultaneously and at a high density. It also describes methods by which a large number of distinct amplified nucleic acid sequences can be monitored at a fast rate and, if desired, in parallel. It also describes methods by which the sequences of a large number of distinct nucleic acids can be determined simultaneously and within a short period of time.

Certain aspects of the present invention were published online by the inventors 19 Mar. 2009 in BMC Genomics 2009, 10:116,doi 10.1186/1471-2164-10-116, incorporated by reference herein for further illustration of experimental protocols.

### BRIEF SUMMARY OF THE INVENTION

The following brief summary is not intended to include all features and aspects of the present invention, nor does it imply that the invention must include all features and aspects discussed in this summary.

In certain aspects, the present invention comprises a method of determining the concentration of DNA molecules in a sample. The sample is preferably a DNA sequencing library, such as is prepared to contain a collection of DNA molecules from a particular sample, where the molecules are prepared for use in a sequencing method or device, as in massively parallel sequencing. A library of nucleic acid molecules to be used in a sequencing project may be quantified, in order to add a proper concentration of nucleic acids to various reaction areas or wells for sequencing. The DNA or other nucleic acid molecules in the sample will have different sequences, and the sequences of the individual molecules may not be known. The method comprises providing a library comprising a plurality of individual DNA molecules, each with a 5′ adapter and a 3′ adapter, said adapters spanning a sequence of interest. The adapters, described more fully below, will have regions of common sequence as between all 5′ adapters and all 3′ adapters. These regions may vary depending on the sequencing methodology to be used. The adapters flank a sequence of interest, i.e., the DNA molecule being sequenced. The method further comprises distributing said individual DNA molecules from the library to a number of individual reaction areas, wherein the percentage of reaction areas containing one or more of the DNA molecules is greater than 0 percent and less than 100 percent. In this distribution, there is a certain random chance that a reaction area may contain 0, 1, or more molecules. The distribution is done, e.g., by dilution of the sample, so that some, but not all of the reaction areas contain DNA molecules. For example, 50%-90% of the reaction areas may be positive for DNA. Or, about 80% of the reaction areas may be positive for a DNA molecule. In this case, it may be calculated that a positive reaction area contains on average about 2 molecules. The method further comprises amplifying the DNA molecules, if present in a reaction area, using a forward primer binding to the 5′ adapter and a reverse primer binding to the 3′ adapter on the single molecule. A number of primer based amplification methods are known, most notably the polymerase chain reaction, PCR. The method further comprises the step of generating a signal in each the reaction area containing amplified molecules. The amplified product may be detected by optical means, namely a fluorescent probe or other molecule which fluoresces as a result of the amplification process. As a result, the number of reaction areas generating a signal is indicative of the quantity of DNA molecules in the sample.

In other aspects of the present invention, the method comprises the steps of (a) obtaining a sample of individual DNA molecules; and (b) ligating or otherwise attaching a 5′ adapter on a 5′ end of each molecule and a 3′ adapter on a 3′ end of each molecule, each 5′ adapter having the same sequence and 3′ adapter having the same sequence. This step is often used in sequencing methods. Also, one then (c) distributes said individual molecules, after said ligating, to a number of individual reaction areas, each reaction area having on average no more than one molecule per area. This step may be part of a digital PCR step. The method further comprises (d) amplifying a single molecule, if present in a reaction area, using a forward primer binding to the 5′ adapter and a reverse primer binding to the 3′ adapter on the single molecule, and (e) generating a signal by means of a probe which binds to a sequence defined by a forward primer or a reverse primer, said signal being dependent upon amplification, whereby the number of reaction areas generating a signal is indicative of the quantity of DNA molecules in the sample. The method may also involve distributing in a microfluidic device adapted for carrying out PCR reactions in individual reaction areas. Such a device would permit proper sequential reactions and thermocycling. Distributing into individual reaction areas may be done by a variety of methods, such as a microfluidic device, a gel, an emulsion, a bead, or a multiwell plate. An individual molecule can be isolated in an emulsion, attached to a bead, or deposited in a well of a multiwell plate. In the case of a gel, an individual nucleic acid molecule is isolated in a location on a gel.

The method may further comprise a step where the forward primer contains a complementary sequence for binding of the probe. This probe binding sequence is not necessarily part of the primer sequence that binds to the template (adapter). The probe binding is contemplated as being part of the detection that a sample molecule was present. The method may further comprise the step of adding forward primer both with and without probe binding complementary sequence during the amplification reaction.

In certain aspects, the method may involve carrying out said PCR where the amplification is done in at least 700 reaction areas, or at least 7,000 reaction areas.

In certain aspects, the method may involve a step where the probe contains a fluorescent molecule and a quencher which are separated during said amplifying to generate fluorescence. The probe may contain 7-12 bases which are complementary to the probe binding site and a fluorescent dye and quencher at opposite ends. The probe may also contain at least one normatural base to increase binding affinity.

In certain aspects, the method may involve a method for sequencing DNA. The sequencing process begins with a library of DNA molecules and comprises obtaining a sample of individual DNA molecules from the library to be sequenced; ligating a 5′ adapter on a 5′ end of each molecule and a 3′ adapter on a 3′ end of each molecule, each 5′ adapter and 3′ adapter having the same sequence; distributing said individual molecules to a number of individual reaction areas, each reaction area having on average no more than one molecule per area; amplifying a single molecule, if present in a reaction area, using a forward primer binding to the 5′ adapter and a reverse primer binding to the 3′ adapter on the single molecule; generating a signal by means of a probe which binds to a sequence defined on a forward primer or a reverse primer, whereby the number of reaction areas generating a signal is indicative of the quantity of DNA molecules in the sample; and sequencing the sample using an amount of DNA determined by the quantity of DNA as determined in step (e).

In certain aspects, the method may involve a method for using a universal template for a probe, said probe being fluorescent, said method comprising a real time PCR reaction, said method being characterized by the use as said probe of a probe having a length of between 8 and 12 bases, at least one of said bases being a normatural base for higher binding to the template.

In certain aspects, the present invention may involve a hydrolysis probe having a sequence complementary to a portion of a PCR primer, said portion being non-complementary to the primer's template. Hydrolysis, or cleavage, of the probe by a polymerase removes a quencher, allowing a fluorescent signal to be generated. In this case, the primer's template region is a sequence in the primer that binds to the molecule to be amplified, as is known in the art.

In certain aspects, the method may involve a kit for quantifying a population of nucleic acid strands, comprising 5′ adapters and 3′ adapters for the nucleic acid strands, each 5′ adapter and 3′ adapter having the same sequence; forward and reverse primers complementary to the 5′ and 3′ adapters, respectively, said forward primer having a non-complementary region for providing a sequence for binding of a labeled probe; and said labeled probe having a fluorescer-quencher pair which provides an optical signal during amplification, said labeled probe further characterized as having between 7 and 15 bases, and having a non-natural base for increasing binding.

### BRIEF DESCRIPTION OF THE DRAWINGS

FIG. 1A-1F is a series of six traces, three from a Nanodrop spectrophotometer (FIG. 1A, FIG. 1C and FIG. 1E), and three from Agilent capillary electrophoresis (FIG. 1B, FIG. 1D and FIG. 1F), representing detection of three trace CHIP 454 sst DNA libraries prepared from 40-60 ng input mouse chromatin by digital PCR. The signals in the electropherograms are molecular weight markers of 15 by and 1500 bp. Three samples are illustrated: IgG (FIGS. 1A, 1B); K27-1 (FIGS. 1C, 1D); and K27-2 (FIGS. 1E, 1F).

FIG. 2 is a photograph of a microfluidic chip showing detection of library molecules by digital PCR, showing an image of 12×768 digital array at assay endpoint. Each grid point corresponds to a nanoliter-scale PCR reaction, with light color points (yellow in original false color image) revealing amplification due to the presence of at least one sequencing library template molecule. There are two columns in the array, each having six, independent, panels (12 total). The panels show indicated dilution series of samples analyzed in part A, allowing accurate absolute quantification of the sample by UT digital PCR. That is, in the top right, library K27 at 1:1000 shows far fewer amplifications than the top left, K27 at 1:100 dilution.

FIG. 2 shows how library quantification is carried out at different dilutions. The user can translate number of spots on chip (single molecules) into molecules per μL. In order to run on a high throughput sequencer, the correct concentration (in molecules/μL) is vital in order to ensure the throughput of the instrument and quality of the sequencing result. For example, one can take the number of positive spots in a panel (e.g., 200), divide by 4.6 μL which is the volume in the panel, and times that by the prepared reaction volume (e.g., 10 ul) and divide it by the input DNA volume (e.g., 1 ul). This is then multiplied by the dilution factor, that is, initial molecule count. The sample may then be diluted to a working concentration of e.g., 4 pM for a Solexa type sequencing, or 2×10⁵ molecules/μL, for a 454 Flx type of sequencing.

FIG. 3 is a bar graph showing coefficient of variation for libraries quantitated by digital PCR and real time PCR (qPCR).

FIG. 4 is a plot showing accurate digital PCR quantitation of 454 libraries from trace amounts (100 pg to 35 ng) of input E. coli genomic or amplicon DNA. Useful numbers of library molecules are recovered.

FIG. 5 is a histogram of frequency of bead enrichment fractions obtained in 454 sample preparation when digital PCR is used as the calibration. The manufacturer's recommended range is 10% to 15%, and the results using titration runs range between 14% and 28%.

FIG. 6 is a histogram showing frequency of mixed fraction from 454 sequencing runs using samples calibrated by digital PCR. The manufacturer specifies the acceptable range to be 20% to 30% and our results using titration runs range between 22% and 35%.

FIG. 7 is a histogram showing cluster density of normalized Solexa sequencing results comparing the percentage of cluster generated per tile using UT-dPCR vs. Standard Quantitation (note normalized to 125,000 clusters per tile). It is expected that users will perform titration using standard quantitation in order to gauge the best dilution of DNA in order to reach the optimal cluster density, once quantification of the library is achieved using the present method.

FIG. 8A-8B is a schematic drawing showing the use of the probes and primers in the present method, where one reaction path is shown in FIG. 8A and another path which starts from the same mixture of primers, probes and DNA template is shown in FIG. 8B.

### DETAILED DESCRIPTION OF THE PREFERRED EMBODIMENT

### Overview

Several of the next generation sequencers are limited in their sample preparation process by the need to make an absolute measurement of the number of template molecules in the library to be sequenced. The practical effects of this compromise performance, both by requiring large amounts of sample DNA and by requiring extra sequencing runs to be performed. The present specification describes quantitation of “454 libraries,” i.e., for use with sequencers from 454 Life Sciences, a Roche Company, e.g., in the FLX System, and prepared according to manufacturer's instructions. Also described is quantitation of “Solexa libraries,” i.e., prepared according to manufacturer's instructions for use with an Illumina/Solexa machine such as a Genome Analyser. These massively parallel sequencing methods and machines depend on large numbers of sequencing reads from a mixture of DNA fragments prepared for the particular sequencing methodology used.

The present method, as exemplified, used digital PCR for sequencing library quantitation and demonstrated its sensitivity and robustness by preparing and sequencing libraries from subnanogram amounts of bacterial and human DNA on the 454 and Solexa sequencing platforms. This assay allows absolute quantitation and eliminates uncertainties associated with the construction and application of standard curves. The digital PCR platform consumes subfemptogram amounts of the sequencing library and gives highly accurate results, allowing the optimal DNA concentration to be used in setting up sequencing runs. This approach reduces the sample requirement more than 1000-fold: from micrograms to less than a nanogram without pre- or post-amplification steps or the associated bias and reduction in library depth. Furthermore, the high accuracy and reproducibility of the measurement allows new libraries to enter bulk runs at the ideal concentration without costly and time-consuming titration techniques.

Some detection chemistries for real-time PCR, such as TaqMan, have the property of counting molecules rather than measuring DNA mass, although the measurements are relative and the methods by which standards are established often tie the real-time PCR quantitation back to sample mass. Digital PCR is a technique where a limiting dilution of the sample is made across a large number of separate PCR reactions such that most of the reactions have no template molecules and give a negative amplification result. In counting the number of positive PCR reactions at the reaction endpoint, one is counting the individual template molecules present in the original sample one-by-one. PCR-based techniques have the additional advantage of only counting molecules that can be amplified, e.g., that are relevant to the massively parallel PCR step in the sequencing workflow. The present examples were generated using Fluidigm's Biomark platform for digital PCR, which has the advantages of low reagent costs and easy setup of 9,180 PCR reactions per chip due to the automated partitioning of nanoliter PCR reactions.

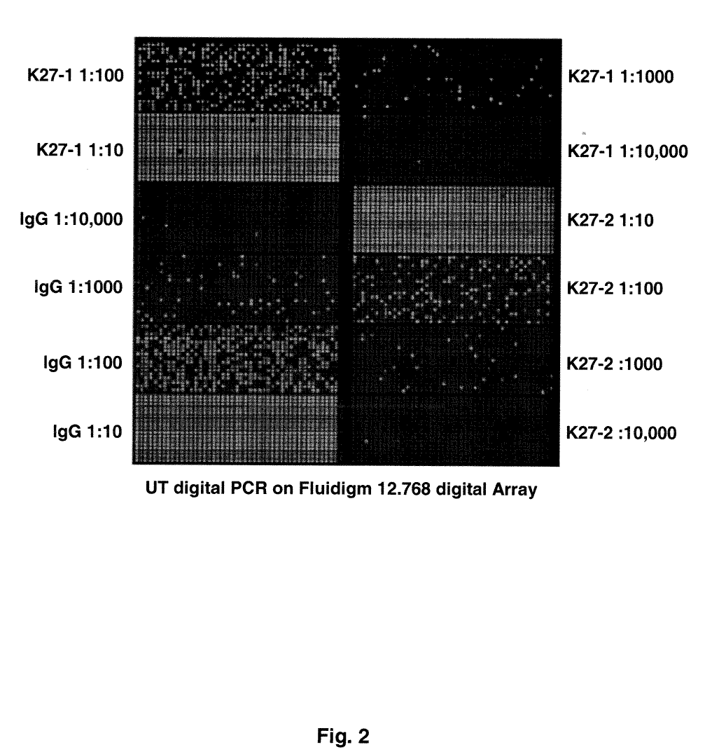

In the present digital PCR-based methods, one distributes molecules from the sequencing library into a number of different reaction areas (well, beads, emulsions, gel spots, chambers in a microfluidic device, etc.). It is important that some reaction areas, but not all, contain at least one molecule. Ideally, each reaction area will contain one or zero molecules. See, Quake et al., “Non-invasive fetal genetic screening by digital analysis,” US 20070202525. In practice, there will be a more or less random distribution of molecules into wells. In the case where a percentage of reaction areas (e.g., 80% is positive, a number of areas will contain one or more molecules, e.g., an average of 2.2 molecules per well. Statistical methods may be used to calculate the expected total number of molecules in the sample, based on the number of different reaction areas and the number of positives. This will result in a calculated concentration of DNA molecules in the sample that was applied to the different reaction areas. A number of statistical methods based on sampling and probability can be used to arrive at this concentration. An example of such an analysis is given in Dube, “Computation of Maximal Resolution of Copy Number Variation on a Nanofluidic Device using Digital PCR (2008),” found at arxiv.org, citation arXiv:0809.1460v2 [q-bio.GN], first uploaded on 8 Sep. 2008. FIG. 2 in this paper sets forth a series of equations that may be used to estimate the concentration of molecules and statistical confidence interval based on the number of reaction areas used in a digital PCR array and the number of positive results. Another example of this type of calculation may be found in U.S. patent application Ser. No. 12/170,414 filed on Jul. 9, 2008. The accuracy of the concentration determination may be improved by using a greater number of reaction areas. One may use approximately, 100-200, 200-300, 300-400, 700 or more reaction areas.

In the examples below, using accurately quantitated amounts of starting material, it is shown that the TaqMan® assay is sensitive, accurate, and robust for PCR-based quantitation of libraries made from as little as 100 pg of starting material. When combined with digital PCR, dependence on a standard sample is eliminated, and the results are sufficiently accurate to allow the elimination of titration techniques, even for samples of low quantity and low quality.

### DEFINITIONS

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well known and commonly used in the art. Certain experimental techniques, not specifically defined, are generally performed according to conventional methods well known in the art and as described in various general and more specific references that are cited and discussed throughout the present specification. For purposes of the clarity, following terms are defined below.

The term “digital PCR” means an amplification (i.e., creation of numerous essentially identical copies) which is carried out on a nominally single, selected starting molecule, where a number of individual molecules are each isolated in a separate reaction area. It is contemplated that numerous reaction areas will be used, to produce higher statistical significance. Each reaction area (well, chamber, bead, emulsion, etc.) will have either a negative result, if no starting molecule is present, or an amplification, for purposes of detection, if the targeted starting molecule is present. By analyzing the number of positive reactions, insight into the number of starting molecules is obtained. A number of methodologies for digital PCR exist. For example, emulsion PCR has been used to prepare small beads with clonally amplified DNA—in essence, each bead contains one type of amplicon of digital PCR. This is further described in Dressman et al, Proc. Natl. Acad. Sci. USA. 100, 8817 (Jul. 22, 2003). Fluorescent probe-based technologies, which can be performed on the PCR products “in situ” (i.e., in the same wells), are particularly well suited for this application. This method is described in detail in Vogelstein PNAS 96:9236, above, and Vogelstein et al. “Digital Amplification,” U.S. Pat. No. 6,440,705, incorporated by reference below contains a more detailed description of this amplification procedure. The polony technique referenced below may also be used in a digital manner. These amplifications may be carried out in an emulsion or gel, on a bead or in a multiwell plate. What is necessary is that one molecule on average or no molecule be present in a number of reactions, such that the number of positive reactions is indicative of the number of molecules present in a sample. Accordingly, it is understood that a large number of emulsions, isolated individual molecules in a gel, beads, wells, etc. are used.

The term “digital PCR” also includes microfluidic-based technologies where channels and pumps are used to deliver molecules to a number of chambers (see e.g., FIG. 2B for illustration of a suitable array of multiple chambers). A suitable microfluidic device is produced by Fluidigm Corporation, termed the Digital Isolation and Detection IFC (integrated fluid circuit). Further description of such a device may be found in U.S. Pat. No. 6,408,878 to Unger, et al., issued Jun. 25, 2002, entitled “Microfabricated elastomeric valve and pump systems.” A suitable device is also described in U.S. Pat. No. 6,960,437 to Enzelberger, et al., issued Nov. 1, 2005 entitled “Nucleic acid amplification utilizing microfluidic devices,” which describes a microfluidic device capable of supporting multiple parallel nucleic acid amplifications and detections. As described in this patent, one exemplary microfluidic device for conducting thermal cycling reactions includes in the layer with the flow channels a plurality of sample inputs, a mixing T-junction, a central circulation loop (i.e., the substantially circular flow channel), and an output channel. The intersection of a control channel with a flow channel can form a microvalve. This is so because the control and flow channels are separated by a thin elastomeric membrane that can be deflected into the flow channel or retracted therefrom. Deflection or retraction of the elastomeric membrane is achieved by generating a force that causes the deflection or retraction to occur. In certain systems, this is accomplished by increasing or decreasing pressure in the control channel as compared to the flow channel with which the control channel intersects. However, a wide variety of other approaches can be utilized to actuate the valves including various electrostatic, magnetic, electrolytic and electrokinetic approaches. Another microfluidic device, adapted to perform PCR reactions, and useful in the present methods, is described in US 2005/0252773 by McBride, et al., published Nov. 17, 2005, entitled “Thermal reaction device and method for using the same.”

Another suitable device which may be adapted for amplification reactions is described in “System for high throughput sample preparation and analysis using column,” U.S. Pat. No. 6,932,939 assigned to BioTrove, Inc.

The term “generating a signal” means a result of a detectable reaction, such as a molecule which is labeled with a dye, such as the fluorescent probe described above, as well as a probe which has a fluorescer and quencher, in which the nuclease activity of the polymerase enzyme used in the amplification causes fluorescence. Another suitable probe which generates an optical signal is a molecular beacon (MB) probe. MB probes are oligonucleotides with stem-loop structures that contain a fluorescent dye at the 5′ end and a quenching agent (Dabcyl) at the 3′ end. The degree of quenching via fluorescence energy resonance transfer is inversely proportional to the 6th power of the distance between the Dabcyl group and the fluorescent dye. After heating and cooling, MB probes reform a stem-loop structure, which quenches the fluorescent signal from the dye. If a PCR product whose sequence is complementary to the loop sequence is present during the heating/cooling cycle, hybridization of the MB to one strand of the PCR product will increase the distance between the Dabcyl and the dye, resulting in increased fluorescence.

The term “hydrolysis probe” means a probe which is used to generate a signal during an amplification reaction as a result of hydrolysis or other cleavage of the probe. It typically involves a homogeneous 5′-nuclease assay (e.g., the nuclease activity of a DNA polymerase used in PCR), since a single 3′-non-extendable (due to phosphorylation) probe, which is cleaved during PCR amplification, is used to detect the accumulation of a specific target DNA sequence. This single hydrolysis probe contains two labels in close proximity to each other: a fluorescent reporter dye at the 5′-end and a (fluorescent or dark) quencher label at or near the 3′-end. When the probe is intact, the fluorescent signal is almost completely suppressed by the quenching label. When the probe is hybridized to its target sequence, it is cleaved by the 5′→3′ exonuclease activity of a polymerase, such as the FastStart Taq DNA Polymerase, which “unquenches” the fluorescent reporter dye. During each PCR cycle, more of the released fluorescent dye accumulates, boosting the fluorescent signal. In the preferred embodiment, the probe binds to a specified strand along its length, as in a Taqman probe. In the preferred embodiment, stem-loop structures, as in Molecular Beacons, may also be used. Black Hole Scorpions, or Amplifluor Direct molecules, combining the primer and probe in one molecule may also be used.

The present probes may be designed according to Livak et al., “Oligonucleotides with fluorescent dyes at opposite ends provide a quenched probe system useful for detecting PCR product and nucleic acid hybridization,” PCR Methods Appl. 1995 4: 357-362. Common dye-quencher pairs are fluorescein and rhodamine dyes. The present probes may be hydrolysis probes, or molecular beacon, scorpion or other probes generating a signal upon amplification.

A wide variety of reactive fluorescent reporter dyes are known in the literature and can be used so long as they are quenched by the corresponding quencher dye of the invention. Typically, the fluorophore is an aromatic or heteroaromatic compound and can be a pyrene, anthracene, naphthalene, acridine, stilbene, indole, benzindole, oxazole, thiazole, benzothiazole, canine, carbocyanine, salicylate, anthranilate, coumarin, fluorescein, rhodamine or other like compound. Suitable fluorescent reporters include xanthene dyes, such as fluorescein or rhodamine dyes, including 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p-(2-benzoxazolyl)phenyl) maleimide; cyanines, such as indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); BODIPYTM dyes; benzoxaazoles; stilbenes; pyrenes; and the like. For further details, see WO/2005/049849, “fluorescence quenching azo dyes, their methods of preparation and use.” As is known in the art, suitable quenchers are selected based on the fluorescer used.

The term “locked nucleic acid” (LNA) means a class of nucleic acids analogues, where the ribose ring is “locked” with a methylene bridge connecting the 2′-O atom with the 4′-C atom (see structure below). LNA nucleosides containing the six common nucleobases (T, C, G, A, U and mC) that appear in DNA and RNA are able to form base-pairs with their complementary nucleosides according to the standard Watson-Crick base pairing rules. Therefore, LNA nucleotides can be mixed with DNA or RNA bases in the oligonucleotide whenever desired. The locked ribose conformation enhances base stacking and backbone pre-organization, this gives rise to an increased thermal stability and discriminative power of duplexes. LNA discriminates single base mismatches under conditions not possible with other nucleic acids. Locked nucleic acid is disclosed for example in WO 99/14226.

The LNA is a non-natural base for increasing binding. Other methods of increasing binding affinity, permitting the use of shorter probes, may be employed. For example, U.S. Pat. No. 5,432,272 has disclosed methods for synthesizing oligonucleotide analogs built from nucleosides carrying nucleobases that can form base pairs using non-standard hydrogen bonding patterns. By using non-standard hydrogen bonding patterns, the number of independently replicating building blocks in an oligonucleotide can be increased from four to six, eight or more, to a maximum of twelve. Other non-natural nucleotides for increasing binding are disclosed in U.S. Pat. No. 5,958,691 to Pieken, et al., issued Sep. 28, 1999, entitled “High affinity nucleic acid ligands containing modified nucleotides.”

The term “sequencing by synthesis” means a method of obtaining a nucleotide sequence from an unknown template molecule which is based on reactions arising from incorporating bases into the template and thereby synthesizing a double stranded DNA from a primed single stranded DNA molecule. The term further refers to such sequencing methods which rely on a sample preparation step wherein adapters are added to sample DNA molecules; and where the DNA molecules are amplified prior to sequencing. As described below, this typically involves massively parallel sequencing and PCR. The term sequencing by synthesis has somewhat different art recognized meanings, as explained in Metzger, “Emerging technologies in DNA sequencing,” Genome Res. 15:1767-1776, 2005. However, in this case, the term refers to a sequencing method, referred to in the art as “Polony Cyclic Sequencing by Synthesis,” in that is uses a “polony” or polymerase-colony for massively parallel amplification of individual DNA molecules in a sample, as further described in Mitra, R. and Church, G. M. (1999), “In situ localized amplification and contact replication of many individual DNA molecules. Nucleic Acids Res. 27(24):e34; pp. 1-6.” In polony Examples of the present sequencing by synthesis are given in Porreca G J, Zhang K, Li J B, Xie B, Austin D, Vassallo S L, LeProust E M, Peck B J, Emig C J, Dahl F, Yuan Gao Y, Church G M, Shendure, J (2007) Multiplex amplification of large sets of human exons. Nat. Methods. 2007 November; 4(11):931-6 (Illumina/Solexa); Margulies, M. Eghold, M. et al. (2005) Genome sequencing in microfabricated high-density picolitre reactors Nature, Sep. 15; 437(7057):326-7 (454/Roche); etc.

The term “UT” is an abbreviation of universal template, in that a probe-binding sequence is appended to one of the PCR primers, as described in detail below. “dPCR” is an abbreviation of digital PCR. “emPCR” is an abbreviation of emulsion PCR. “qPCR” is an abbreviation of quantitative PCR.

The numbers 5′ and 3′ are used in their conventional sense. The numbers refer to the numbering of carbon atoms in the deoxyribose, which is a sugar forming an important part of the backbone of the DNA molecule. In the backbone of DNA the 5′ carbon of one deoxyribose is linked to the 3′ carbon of another by a phosphate group. The 5′ carbon of this deoxyribose is again linked to the 3′ carbon of the next, and so forth. The same terminology is used for RNA.

### General Methods and Materials

The present methods involve a method of quantifying nucleic acid (RNA or DNA) molecules in a sample, as where on needs to know the concentration of DNA molecules in a sequencing library in order to deliver the molecules to a sequencing device in the most efficient way. The method comprises obtaining a sample of individual DNA molecules, e.g., a portion of the sequencing library; and ligating a 5′ adapter on a 5′ end of each molecule and a 3′ adapter on a 3′ end of each molecule, each 5′ adapter and 3′ adapter having the same sequence. This step is presently undertaken as part of a process used in a number of massively parallel sequencing methodologies, which can result for example, in approximately 3 to 5 million reads with 2 to 3 million mapable unique fragments per sample. The present quantification method further comprises the step of distributing said individual molecules, after said ligating, to a number of individual reaction areas, each reaction area having on average no more than one molecule per area; amplifying a single molecule, if present in a reaction area, using a forward primer binding to the 5′ adapter and a reverse primer binding to the 3′ adapter on the single molecule; and generating a signal by means of a probe which binds to a sequence defined by a forward primer or a reverse primer, said signal being dependent upon amplification. By using this method, whereby the probe gives a signal in each reaction area, and only in each reaction area where a nucleic acid molecule was present, the number of reaction areas generating a signal is indicative of the quantity of DNA molecules in the sample.

To overcome the challenge of probe design for templates of random sequence, which is necessary when the probe hybridizes to an unknown sequence, primers were prepared which bind to the adapter molecules, and, furthermore, utilized a portion of a primer (preferably the forward primer) as a universal template (UT). That is, a probe-binding sequence is appended to one of the PCR primers. The same probe binding sequence, and probe, can be used with many different primers. This approach utilizes certain aspects of the Zhang et al. method referenced above, namely a universal template probe, with significant differences. For example, to speed reaction times, the published 20 by UT probe-binding region of Zhang et al. was replaced with an 8 by sequence target for a probe containing a locked nucleic acid nucleotide such as Roche's UPL (Universal Probe Library) probes. The shorter amplicon-probe interaction length allows the reduction of PCR run times from 2.5 hours to less than 50 minutes. The probe must bind to the primer with greater energy than the primer binds to the template. The 8 bp The underlined sequence GGC GGC GA (SEQ ID NO:11) in Table 6 below represents the presently exemplified 8 mer universal template (UT) portion of the primer (where the binding portion is generally about 18-20 bases in addition to the UT portion). That is, the UT primer has, in addition to a sequence binding to the universal adapter attached to the template DNA, a sequence (UT portion) which will hybridize to the signal generating probe.

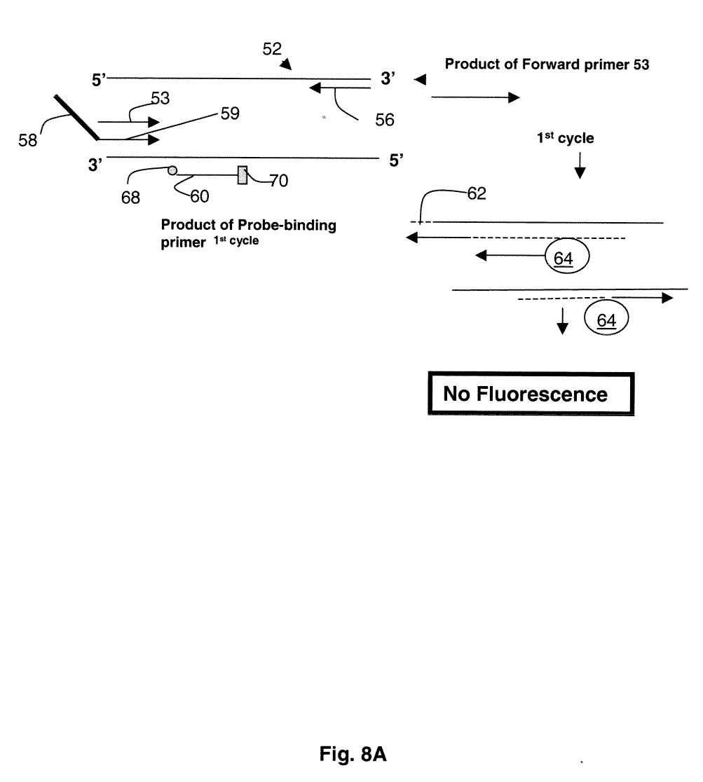

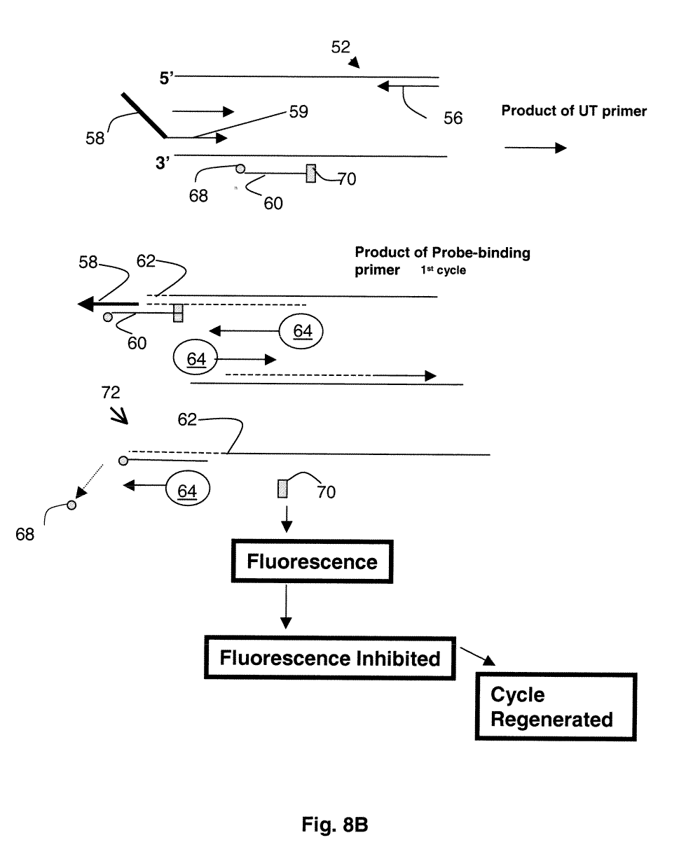

Referring now to FIGS. 8A and B, the template DNA to be quantitated 52 is shown as double stranded, with a 5′ and a 3′ end for each complementary strand. The strands are amplified with primers, 56, 53, 59, for each strand. Since primers 53 and 59 are both forward primers, they compete for binding to the template. That is, two primers are shown for the bottom strand: 53 and 59. One primer, 59, shown as a forward primer, is longer than the other, having a template binding portion 59 and, additionally, a UT portion 58. The UT portion 58 does not bind to the template and has a short sequence comprising, e.g., the 8 bases mentioned above. These 8 bases are complementary to a UT probe 60, just below the bottom strand, for purposes of illustration. The UT probe 60 has at its ends a fluorescent label 70 and a quencher 68. All three primers are designed to hybridize only to the sequencing adapters, added on to the ends of the DNA molecules that are to be the subject of the massively parallel sequencing. The sequencing adapters will have the same sequence for all 5′ adapters and all 3′ adapters. They are typically provided by the sequencing manufacturer. Thus, the primers 56, 53, 59 will hybridize to, and allow amplification and detection of, all molecules in the library sample being analyzed. The probe hybridizes to one of the primers. The probe preferably contains a locked nucleic acid. As is known, the amplification process comprises a reaction which causes the UT probe to be digested, or hydrolyzed, typically due to a nuclease activity of the polymerase used in the amplification process. Cleavage of the probe results in separating the quencher from the fluorescer, and therefore causing a detectible optical signal which is increased as the number of amplifications increases.

The method also uses a second forward primer 53 which does not contain a UT portion. Use of this primer serves to drive the polymerization forward. This non-UT primer is preferably about 50% of the forward primer mixture. Thus, the preferred amplification uses both a UT primer and an identical primer without the UT in the same reaction. Some templates will not have probe binding sites because they were created with a primer 53 that lacks such a site. This forward primer 53 feeds the efficiency of the reaction. This is thought to be because the UT-binding primer 59 is kinetically unstable, while the non-UT primer 53 is more efficient in binding, thereby giving time for the UT-binding primer 59 to make more probe binding template on the complementary strand.

As further shown in FIG. 8A, the use of forward primer 53, i.e., amplification by forward primer 53, without a UT region, results in a first cycle amplification and an elongated template at 62, resulting from primer overhang. These products will not fluoresce. The portion of the reaction pathway shown in FIG. 8B, on the other hand, generates a fluorescent signal which increases as the number of amplifications increases the number of template molecules which bind probes.

In the portion of FIG. 8B labeled “Product of Probe-binding primer,” it can be seen that the UT probe 60 binds to the portion of a newly synthesized complementary strand that is derived from the primer sequence 58 in the UT binding primer. As can be seen, two molecules of a DNA polymerase 64 create two new strands, as in conventional PCR, the new strands being illustrated as dashed lines. In the next cycle, as shown at 72, the polymerase, which has an exonuclease activity, cleaves the UT probe 60, releasing the quencher 68 and the fluorescent label 70. The release of the fluorescent label 70 releases the inhibitory effect of the quencher 68, which is no longer close enough to inhibit fluorescence. Thus, as shown, fluorescence occurs. Fluorescence is inhibited by the quencher until it binds to a template strand and is digested by exonuclease activity during the amplification process.

Since more molecules will be present as the amplification progresses, there will be an exponential increase in fluorescence until a plateau and endpoint are reached. In the real time PCR step, this measurement of the rate of increase of fluorescence may be used to quantitate the starting concentration of template DNA. In the digital PCR analysis, the rate of increase of fluorescence need not be measured—the result is binary for either the presence or absence of a template. It should be noted that the present assay is designed to measure all DNA available in the library for sequencing, because the sequencing step employs adapters that are attached to each DNA molecule and have the same sequence. It could also be used to measure only certain sequences suspected of being in the sample, where only 5′ and 3′ portions, but not the intermediate portion, are known

In a preferred method, a first reaction such as shown in FIG. 8A-B is carried out in the real-time mode (with a calibration standard), without a digital analysis, to range the library concentration so that an appropriate dilution can be made for absolute quantitation by UT-digital PCR.

The present primers are designed to bind to the adapters on the ends of the DNA strands; one primer also defines an optical probe (UT probe) binding sequence. The UT probe binds to the complementary strand that has been PCR'ed, i.e., an additional sequence on the strand created by synthesis of a strand extending the UT primer, because the UT primer contains additional sequence which overhangs the end of the template DNA. UT probes (designed for other purposes) can be obtained from commercial sources, as the present probe was obtained from Roche. In the presently illustrated embodiment, the UT primer is a forward primer, i.e., it binds upstream of where the reverse primer binds, and extends across the gene of interest. In the present process, the terms “forward primer” and “reverse primer” are used for convenience, since the sequence of interest (i.e., being amplified) is arbitrary. That is, in PCR, the DNA polymerase synthesizes a new DNA strand complementary to the DNA template strand by adding dNTPs that are complementary to the template in 5′ to 3′ direction, condensing the 5′-phosphate group of the dNTPs with the 3′-hydroxyl group at the end of the nascent (extending) DNA strand. It operates on both strands similarly, using both primers. Variations on the exemplified process are possible. Internal controls using other primers can be used, or other forms of multiplex PCR or DNA amplification can be used, such as rolling circle amplification Since adapters are ligated to both ends of template DNA, UT primer/probes can be used at either or both DNA template ends. Also, as stated various UT primers can be designed, which may contain a signal generating probe portion as part of the primer.

Primers can be prepared by a variety of methods including but not limited to cloning of appropriate sequences and direct chemical synthesis using methods well known in the art (Narang et al., Methods Enzymol. 68:90 (1979); Brown et al., Methods Enzymol. 68:109 (1979)). Primers can also be obtained from commercial sources such as Operon Technologies, Amersham Pharmacia Biotech, Sigma, and Life Technologies. The primers can have an identical melting temperature. The lengths of the primers can be extended or shortened at the 5′ end or the 3′ end to produce primers with desired melting temperatures. Also, the annealing position of each primer pair can be designed such that the sequence and, length of the primer pairs yield the desired melting temperature. The simplest equation for determining the melting temperature of primers smaller than 25 base pairs is the Wallace Rule (Td=2(A+T)+4(G+C)). Computer programs can also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering. The TM (melting or annealing temperature) of each primer is calculated using software programs such as Oligo Design, available from Invitrogen Corp.

The annealing temperature of the primers can be recalculated and increased after any cycle of amplification, including but not limited to cycle 1, 2, 3, 4, 5, cycles 6-10, cycles 10-15, cycles 15-20, cycles 20-25, cycles 25-30, cycles 30-35, or cycles 35-40. After the initial cycles of amplification, the 5′ half of the primers is incorporated into the products from each loci of interest, thus the TM can be recalculated based on both the sequences of the 5′ half and the 3′ half of each primer. Any DNA polymerase that catalyzes primer extension and has exonuclease activity can be used including but not limited to E. coli DNA polymerase, Klenow fragment of E. coli DNA polymerase 1, T7 DNA polymerase, T4 DNA polymerase, Taq polymerase, Pfu DNA polymerase, Vent DNA polymerase, bacteriophage 29, REDTaq™. Genomic DNA polymerase, or sequenase. Preferably, a thermostable DNA polymerase is used. A “hot start” PCR can also be performed wherein the reaction is heated to 95° C. for two minutes prior to addition of the polymerase or the polymerase can be kept inactive until the first heating step in cycle 1. “Hot start” PCR can be used to minimize nonspecific amplification. Any number of PCR cycles can be used to amplify the DNA in the digital amplification process, including but not limited to 2, 5, 10, 15, 20, 25, 30, 35, 40, or 45 cycles.

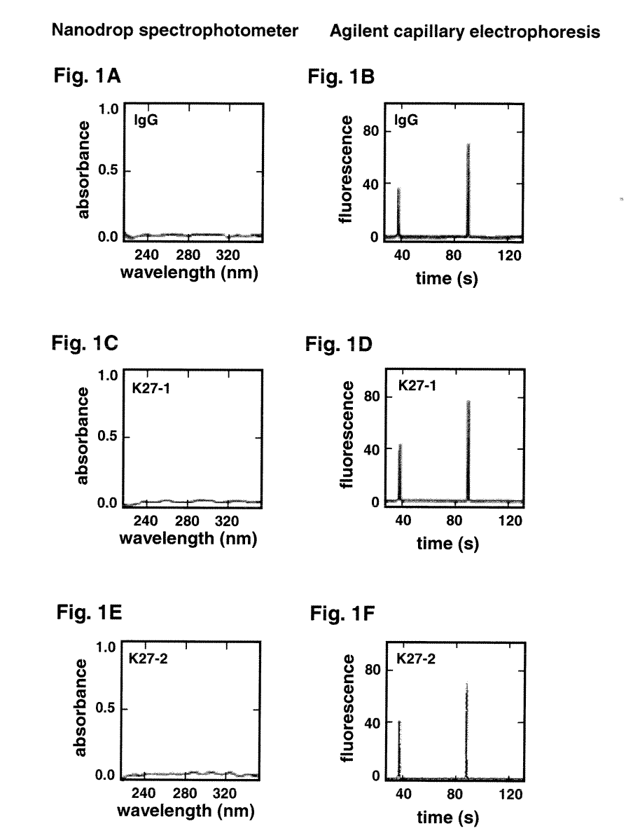

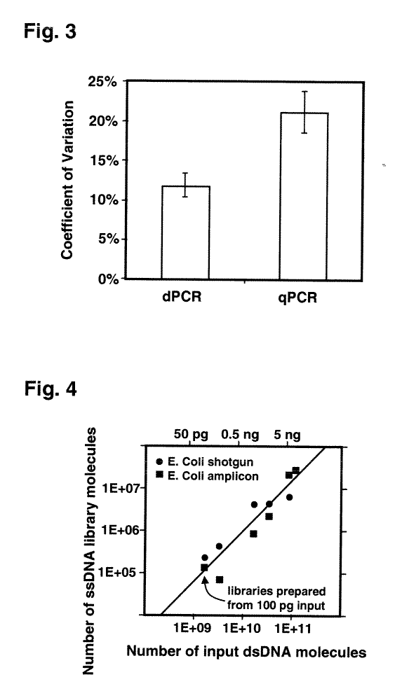

As shown by the comparisons presented below, digital PCR, where nucleic acid molecules in a library are counted, gives an absolute, calibration-free measurement of the concentration of amplifiable library molecules, with a lower coefficient of variation than a real-time PCR measurement with an ideally prepared standard curve, as shown by the plots of calibration in FIGS. 3 and 4. FIGS. 3 and 4 show the reproducibility of the present assays. In FIG. 3, shows that the coefficient of variation for replicate analyses by digital PCR was significantly lower than for quantitative PCR. FIG. 4 shows results from E coli libraries from amplicons (squares) or shotgun genomic sequencing (circles). The input quantity is plotted against yield and shown to scale linearly. More than thirty 454 libraries sequenced without a single titration run over a five month period using the digital PCR quantitation method all gave ideal emPCR and enrichment/sequencing results with a very narrow range of DNA to bead ratios, typically 0.1 DNA per bead, despite dramatic differences in source, type, molecular weight, and quality. FIGS. 1A-F show that libraries that were undetectable with standard methods were quantified using the present methods, as illustrated in FIG. 2. FIG. 2 shows the number of spots in different panels and is summarized below, where the number of + signs indicates an approximation of the number of lit (positive) spots:

| | |
|---|---|
| K27-1 1:100 | ++ |
| K27-1 1:10 | ++++ |
| IgG 1:10,000 | + |
| IgG 1:1,000 | ++ |
| IgG 1:100 | +++ |
| IgG 1:10 | +++++ |
| K27-1 1:10000 | + |
| K27-1 1:10,000 | − |
| K27-2 1:10 | +++++ |
| K27-2 1:100 | ++ |
| K27-2 1:1,0000 | + |
| K27-2 1:10,000 | − |

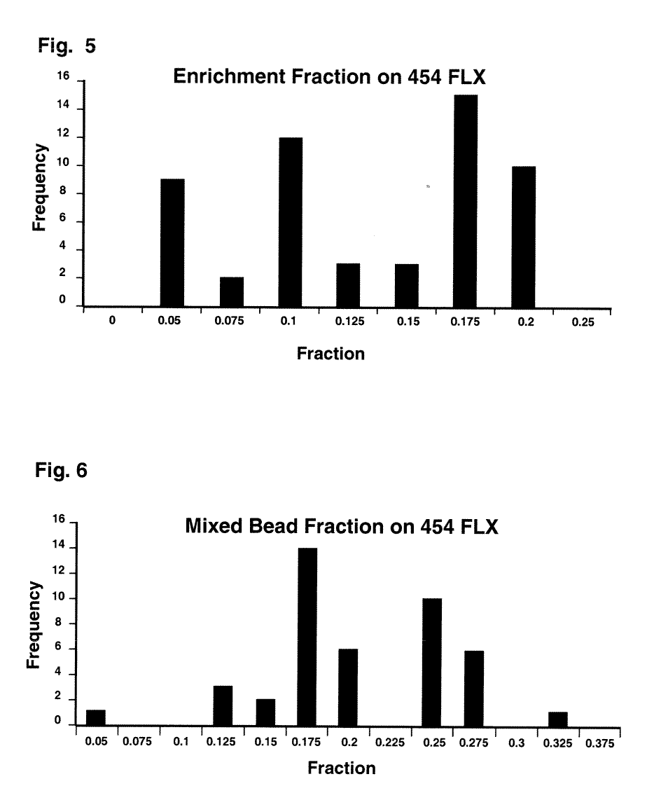

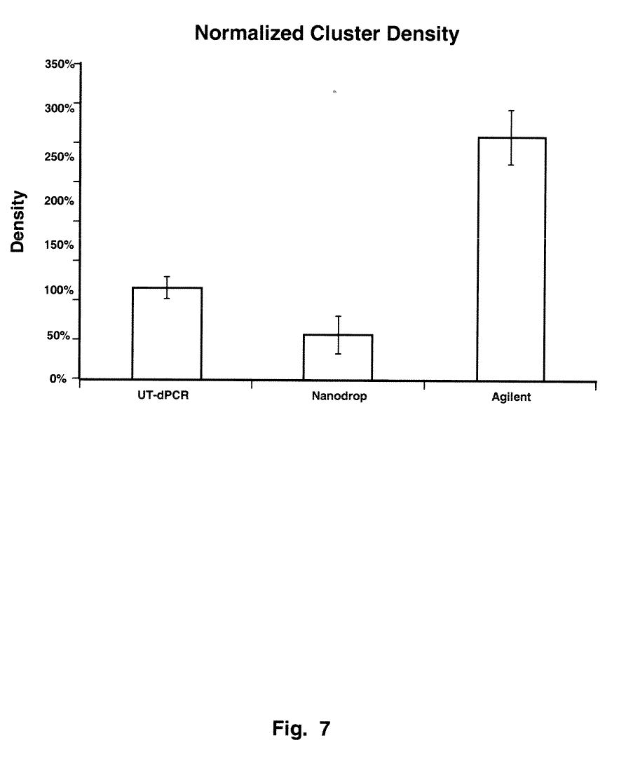

Referring now to FIG. 5, twelve 454 libraries were assayed with six to eight replicates on both UTdPCR and UT-qPCR. UT-qPCR calibrated using a library quantitated by digital PCR. The Coefficient of Variation for dPCR is significantly lower than that for qPCR.

The present probes may be designed in a manner similar to the primers. The sequence will correspond to the sequence of the template created by the elongated primer with the UT sequence. The present UT probes will be short, e.g., 7-10 bases, and are designed to have higher melting temperatures by the incorporation of one or more locked nucleic acids (LNA)

### Examples

To demonstrate the utility of digital PCR in preparing DNA libraries from small amounts of starting material, twelve libraries were created from starting amounts of E. coli genomic DNA from 35 ng to as low as 500 pg. Six of the libraries were constructed with E. coli template DNA (with prior dilution before construction of library) with the standard 454 shotgun protocol with molecule barcodes called MIDs (or Multiplex IDentifiers). Six more DNA libraries of the same quantities were prepared from an E. coli amplification product (of ˜500 bp). The DNA libraries were quantified via dPCR as described. Table 2 shows results from samples TS-1 through TS-12 (E. coli) plus mouse, another bacterium (A. longum) and human samples. Sample pX is a plasma DNA sample from a patient. The designation is random and does not refer to any chromosome, since it's a shotgun sequencing sample meaning everything (entire genomic region) was sequenced.

TABLE 2
Trace Microbial/Human library Construction (Amplicon/Shotgun formats).

| | | | | | ssDNA | | | | |
|---|---|---|---|---|---|---|---|---|---|
| | | | Mean | | library | | | | |
| | | | Library | | (total | | | | |
| | | | frag. | Input | molecules | dPCR | | | |
| Sample | input | | size | (total | by UT- | replicate | | | |
| ID | (ng) | | (Bp) | molecules) | dPCR) | CV | Recovery % | Type | Organism |
| TS-1 | 35 | ng | 500 | 1.17 × 1011 | 2.61 × 107 | 7.07% | 0.022% | Shotgun | E. coli |
| TS-2 | 25 | ng | 500 | 8.37 × 1010 | 6.15 × 106 | 8.24% | 0.007% | Shotgun | E. coli |
| TS-3 | 10 | ng | 500 | 3.35 × 1010 | 4.20 × 106 | 7.71% | 0.013% | Shotgun | E. coli |
| TS-4 | 5 | ng | 500 | 1.67 × 1010 | 4.08 × 106 | 5.65% | 0.024% | Shotgun | E. coli |
| TS-5 | 1 | ng | 500 | 3.35 × 109 | 4.08 × 105 | 16.84% | 0.012% | Shotgun | E. coli |
| TS-6 | 500 | pg | 500 | 1.67 × 109 | 2.31 × 105 | 5.58% | 0.014% | Shotgun | E. coli |
| TS-7 | 35 | ng | 400 | 1.17 × 1011 | 2.67 × 107 | 8.55% | 0.023% | Amplicon | E. coli |
| TS-8 | 25 | ng | 400 | 8.37 × 1010 | 2.11 × 107 | 8.50% | 0.025% | Amplicon | E. coli |
| TS-9 | 10 | ng | 400 | 3.35 × 1010 | 2.15 × 106 | 4.90% | 0.006% | Amplicon | E. coli |
| TS-10 | 5 | ng | 400 | 1.67 × 1010 | 8.49 × 105 | 7.10% | 0.005% | Amplicon | E. coli |
| TS-11 | 1 | ng | 400 | 3.35 × 109 | 8.67 × 104 | 21.90% | 0.003% | Amplicon | E. coli |
| TS-12 | 500 | pg | 400 | 1.67 × 109 | 1.31 × 105 | 7.60% | 0.008% | Amplicon | E. coli |
| IgG | 43 | ng* | 180 | 4.38E+11 | 3.24 × 106 | 17.61% | 0.001% | Shotgun | Mus |
| | | | | | | | | | Musclus |
| K27-1 | 60 | ng* | 180 | 6.11E+11 | 1.87 × 106 | 9.40% | 0.001% | Shotgun | Mus |
| | | | | | | | | | Musclus |
| K27-2 | 60 | ng* | 180 | 6.11E+11 | 1.49 × 106 | 5.65% | 0.001% | Shotgun | Mus |
| | | | | | | | | | Musclus |
| Ace | 723 | ng | 550 | 2.42 × 1012 | 3.63 × 108 | 6.10% | 0.015% | Shotgun | A. longum |
| pX | 60 | ng* | 180 | 6.11 × 1011 | 9.06 × 106 | 4.60% | 0.001% | Shotgun | Homo |
| | | | | | | | | | sapiens |

In Table 2, * indicates DNA samples obtained from chromatin immunoprecipitation (ChIP) experiments. 2000 mouse cells were used for each experiment. The amount of DNA used for 454 library preparation is estimated by assuming 6 pg per well and ˜10% of the genome captured by a typical ChIP experiment. As shown in FIG. 1, with regard to samples IgG, K27-1 and K27-2, the input was undetectable by Nanodrop and Agilent Bioanalyzer. Sample pX was quantified by digital PCR using human specific primers at a unique locus, assuming 6.6 pg per cell equivalent. Other organism specific primers can be used for first approximation testing. The input for library preparation was undetectable by Nanodrop and Agilent Bioanalyzer. The results from FIG. 2 and Table 2 show that enough library DNA can be obtained from 500 pg of genomic (shotgun) or amplicon DNA to obtain more than 100,000 enriched beads for sequencing. All twelve trace libraries were sequenced in a full run of our GS FLX 454 DNA pyrosequencer. In total, 18 million raw bases were sequenced from the trace shotgun libraries and 37.8 million raw bases were sequenced from the amplicon libraries. 69.16% of the shotgun reads mapped back to E. coli, and 99.17% of the amplicon reads mapped to the E. coli template material. Specifically, in the case of the library made from 500 pg of E. coli 16S amplicon, half of the resulting library was used for sequencing. 14.0 million raw bases were obtained in 55,206 reads with 99.02% of the reads mapping back to the template, indicating that almost 30 Mbp can be obtained from a library of 131,000 molecules prepared from 500 pg input material. Similarly, half of the 1 ng E coli amplicon library gave 10.9 million raw bases in 43,217 reads with 99.17% mapping. The 500 pg E coli shotgun library gave 5.7 million raw bases in 26,812 reads (69.9% mapping), while the 1 ng E coli shotgun library gave 6.0 million raw bases in 28,730 reads (69.9% mapping).

TABLE 3
Trace library Generation & Sequence results Solexa

| | | DNA Library | Average number | | |
|---|---|---|---|---|---|
| | | (total molecules | of clusters | Total | % mapping |
| Solexa | Input | by UT- | generated per | number | to Human |
| Libraries | (ng) | dPCR)*/ul | tile | of reads | ref* |
| Plasma | 3.2 | 1.07E+11 | 115998 | 11599833 | 51.46 |
| DNASample 1 | | | | | |
| Plasma DNA | 3.6 | 7.88E+10 | 114548 | 11454876 | 52.66 |
| Sample 2 | | | | | |
| Plasma DNA | 2.7 | 7.17E+10 | 118516 | 11851612 | 56.05 |
| Sample 3 | | | | | |
| Plasma DNA | 2.6 | 6.03E+10 | 150414 | 15041417 | 49.67 |
| Sample 4 | | | | | |
| Plasma DNA | 5.6 | 7.17E+10 | 119104 | 11910483 | 56.13 |
| Sample 5 | | | | | |
| Plasma DNA | 2.4 | 7.23E+10 | 120974 | 12097478 | 55.39 |
| Sample 6 | | | | | |
| Whole Blood | 2.1 | 6.30E+10 | 151201 | 15120171 | 50.52 |
| Genomic DNA | | | | | |
| Sample | | | | | |

A similar UT-dPCR assay was designed to quantify Solexa sequencing libraries. Solexa libraries were prepared from human plasma DNA or whole blood genomic DNA using starting materials between 2-6 ng. The concentration of libraries were determined by UT-dPCR and diluted accordingly. The final concentration of template being loaded onto the sequencing flow cell was 4 pM for all samples. Consistent cluster density between ˜110,000 to 150,000 clusters per tile were achieved on the Genome Analyzer II, a range that is deemed optimal by the manufacturer. The total number of reads yielded was ˜11 to 15 million per lane (Table 4). The samples were also quantitated on the Agilent Bioanalyzer and NanoDrop spectrophotometers. Had the dilutions been determined based on these standard techniques, they would have yielded cluster densities too high and too low by factors of two, respectively.

In an earlier shotgun sequencing run 2,400,000 sstDNA fragments (or 0.71 pg amplifiable DNA) from an Acetonemia longum shotgun library DNA (prepared according to the standard library preparation method from 723 ng of genomic DNA) were used. From these molecules, accurately and reproducibly quantitated by digital PCR, 74% of the beads loaded gave useful 454 sequence data (4.13% ‘mixed’ reads and 4.28% ‘dot’ reads), to yield 67 Mbp in 278,181 reads on one large PTP region. Together with 38 Mbp from another run, 105.6 Mbp of very high quality data was obtained without any titration techniques, 104.3 Mbp of which assembled under Newbler to give coverage of the ˜5 Mbp Acetonemia longum genome with N50 contig size greater than 50,000 bp. Based on the preliminary results from the trace E coli library preparations, it is feasible to combine-the streamlined workflow (no titration runs) together with the sensitive and accurate library quantitation (by digital PCR) to make possible rapid, efficient, and direct sequencing of picogram DNA samples.

TABLE 4
Trace library sequence results 454 Flx

| | | | | | | | | % |
|---|---|---|---|---|---|---|---|---|
| | | | | | | | Average | mapping |
| | | | | Proportion | Raw | Number | read | to |
| Sample | | Library | input | of library | bases | of | length | template/ |
| ID | Organism | Type | (ng) | sequenced | (Mbp) | reads | (bp) | assembling* |
| TS-5 | E. coli | Shotgun | 1.0 ng | 1.0 | 6.0 | 28,730 | 210.5 | 69.9% |
| TS-6 | E. coli | Shotgun | 0.5 ng | 1.0 | 5.7 | 26,812 | 212.5 | 69.9% |
| TS-11 | E. coli | Amplicon | 1.0 ng | 0.5 | 10.9 | 43,217 | 252.5 | 99.2% |
| TS-12 | E. coli | Amplicon | 0.5 ng | 0.5 | 14.0 | 55,206 | 253.6 | 99.0% |
| pX | Human | Shotgun | 60 ng | .1 | 42 | 244,010 | 172.6 | 64.6% |
| Ace | A. longum | Shotgun | 723 ng | 0.005 | 67.0 | 278,181 | 240.9 | 98.8%* |

The extreme sensitivity of real-time and digital PCR eliminate quantitation as the material-limiting step in the sequencing workflow, bringing greater focus to library preparation procedures as the most limiting step in bringing trace samples onto the sequencer. It is natural to expect that library preparation procedures developed with the capacity to handle up to five micrograms of input are far from optimal with respect to minimizing loss from nanogram or picogram samples. Library preparation procedures optimized for trace samples with reduced reaction volumes and media quantities, possibly formatted in a microfluidic chip, have the potential to dramatically improve the recovery of library molecules, allowing preparation of sequencing libraries from quantities of sample comparable to that actually required for the sequencing run, e.g., close to or less than one picogram.

While TaqMan® hydrolysis probes were used here, a multiplicity of detection technologies, including molecular beacon and hybridization, AmpliFluor, scorpion (including the three-oligo ‘scorpions’ format) and LUX probes, are compatible with the universal template approach adopted here, as is the use of modified probe chemistries including LNA (used here), minor-groove binders, PNA, and hydrolysis-resistant and extension-blocking nucleotides.

The digital PCR-based assay, as described in the examples below, was used to quantitate 454 and Solexa sequencing libraries, and, as a result, valid sequence was obtained from a varied collection libraries prepared from hundreds of picograms of starting materials. Digital PCR quantitation is sufficiently accurate in counting amplifiable library molecules to justify elimination of titration techniques as well as the associated cost and time involved. The method is also hundreds of millions of times more sensitive than traditional means of library quantitation, and allows the sequencing of libraries prepared from tens to hundreds of picograms of starting material, rather than the micrograms of DNA required by the manufacturers' protocols. The reduced sample requirement enables the application of next-generation sequencing technologies to minute and precious samples without the need for additional amplification steps, which can severely reduce the diversity of the sequencing library and distort the true distribution of reads.

### Experimental Protocols

Sample generation and Sequencing Library Preparation: The DNA samples for 454 Flx sequencing for trace E. coli shotgun or amplicon were extracted/isolated for mid-log phase K12 over night cultures using Qiagen's DNeasy Tissue & Blood kit then further purified using Qiagen QIAquick PCR purification kit following standard manufactures protocol. The trace E. coli amplicons were generated from K12 specific 16s rRNA PCR following standard protocols generating a uniform 400 by fragment. For Roche 454 Flx library preparation standard library shotgun protocol was followed with small adjustments; trace E. coli amplicons, human sample pX were not nebulized, for each mini-elute column purification step 0.01% Tween-20 was added to the elution buffer during each elution, the final elution volume was 30 μl (for the single strand template (sst) library) it contains 0.05% Tween-20 in 1×TE for long-term storage. The sst library was aliquotted after use and diluted ten fold to reduce library degradation. Solexa libraries were prepared from total DNA extracted from human plasma or whole blood using Qiagen's DNA Blood Mini Kit or Machinerey-Nagel's NucleoSpin Plasma Kit according to manufacturers' protocols. Solexa libraries were generated following standard protocol with small adjustments: all ligated products were used for 18-cycle PCR enrichment; no nebulization was performed on plasma DNA samples since they were fragmented in nature (average ˜170 bp); whole blood genomic DNA sample was sonicated to produce fragments between 100-400 bp; no gel extraction was performed and no Sanger sequencing was used to confirm fragments of correct sequence. Solexa libraries were purified and eluted in 50 μl buffer EB.

Standard creation for UT-qPCR for the Stratagene®Mx3005 Quantitative real time PCR device: After sequencing library preparation, UT-qPCR was used to gauge the general dilution factor that was used for UT-dPCR. For testing purposes and to gauge the correct dilution, a standard library was created, quantitated on UT-dPCR, then serially diluted for standard creation for UT-qPCR. In order to ensure uniform amplification among various libraries the fragment length distribution of the standard matched the library that was generated. To maintain the standard over time, the library was cloned into pCR2.1 (Invitrogen) and then transformed into DH5α cells. Plasmids containing library standard were harvested from mid-log phase DH5α cells and then further isolated using Qiagen's QIAprep Spin Miniprep kit. The resulting plasmids were digested using EcoRI, then gel purified and cleaned up using Qiagen's QIAquick PCR purification kit. Calibration of the UT-dPCR of the standard was conducted on a regular basis.

UT-qPCR quantitation on the Statagene® Mx3005: Validated standards were diluted in ten-fold increments to the dynamic range of 1015-103 molecules/μl. Standards were assayed in triplicate in order to obtain standard deviation/relative coefficient of variation. Each library (454 or Solexa) was diluted ten-fold, and assayed with twelve replicates in order to obtain standard deviation/relative coefficient of variation. Relative coefficient of variation normalizes the UT-qPCR/UT-dPCR measurement of dispersion within a probability distribution.

UT-dPCR quantitation on microfluidic PCR system (Fluidigm's BioMark): For all libraries (Solexa or 454), UT-qPCR was first performed on aliquotted libraries in order to estimate the dilution factor for UT-dPCR. That is, the process may involve an initial step of carrying out a standard quantitative PCR reaction on the library. The libraries were diluted to roughly 100-360 molecules per μl before running on the Fluidigm's Digital Array microfluidic chip. The concentration that yielded 150-360 amplified molecules per panel was chosen for technical replication. Six replicate panels on the digital chip were assayed in order to obtain absolute quantitation of the initial concentration of library. The diluted samples having relative Coefficient of variation (between replicates) within 9-12% (or lower) was used for emPCR (emulsion PCR). Solexa libraries: quantitative qPCR using human specific primers were first performed to estimate the dilution factor required for carrying out UT-dPCR. The final dilution yielded ˜150-360 amplified molecules per panel. Reagents used for all UT-qPCR/UT-dPCR assays consisted of final concentration of 1× Universal Taqman Probe Master Mix (Roche) 200 nM forward primer, 200 nM UT binding primer, 400 nM reverse primer and 350 nM UPL (Universal Probe Library) #149 (Roche). The primer and probe sequences and the thermal cycling parameters are presented in Tables 6 and 5, respectively.

emPCR/Bridge PCR & Sequencing: 454 sequencing was performed according to manufacturer's protocol. No titration or traditional sequencing was used to confirm ratios of DNA, sequence or length. The best DNA:bead ratio obtained from UT-dPCR (digital PCR) quantitation ranged between 0.025-0.3. This gave on average the 10-15% bead recovery and the (lowest mixed sequencing signal. Mixed reads in 454 sequencing is defined when four nucleotide flows are positive for a given read on the sequencer resulting in a mixed signal. For Solexa sequencing the libraries were first diluted to 10 nM according to the concentration determined by digital PCR. The average dilution factor was 10-20. Diluted libraries were denatured with 2N NaOH and then diluted to a final concentration of 4 pM. The templates were loaded onto flow cells. Cluster generation was performed according to the manufacturer's instructions. Sequencing was carried out on the Genome Analyzer II. No titration run was performed.

TABLE 5
Thermocycling Parameters for UT-qPCR/UT-dPCR

| | Standard Adapters 454 | MIDs/Paired-end | MIDs/Paired-end | Solexa |
|---|---|---|---|---|
| | UT-dPCR & UT-qPCR | UT-qPCR | UT-dPCR | UT-dPCR |
| Hot Start | 95 C., 3 mins | 95 C., 3 mins | 95 C., 3 mins | 95 C., 10 mins |
| Denaturation | 94 C., 30 secs | 95 C., 3 secs | 95 C., 15 secs | 95 C., 15 secs |
| Annealing | 60 C., 30 secs | 65 C., 30 secs | 65 C., 30 secs | 60 C., 1 min |
| Extension | 72 C., 45 secs | — | — | — |
| Cycle | 40 | 40 | 40 | 40 |

TABLE 6
Primer/probe list for UT-qPCR/UT-dPCR

Primers for Standard 454 libraries:

| | | |
|---|---|---|
| Forward: | SEQ ID NO: 1 | 5′-CCATCTCATCCCTGCGTGTC-3′ |
| Reverse: | SEQ ID NO: 2 | 5′-CCTATCCCCTGTGTGCCTTG-3′ |
| UTBP-1: | SEQ ID NO: 3 | 5′-GGCGGCGACCATCTCATCCCTGCGTGTC-3′ |

Primers for 454 MID/Paired end libraries:

| | | |
|---|---|---|
| Forward: | SEQ ID NO: 4 | 5′-GCCTCCCTCGCGCCATCAG-3′ |
| Reverse: | SEQ ID NO: 5 | 5′-GCCTTGCCAGCCCGCTCAG-3′ |
| UTBP-2: | SEQ ID NO: 6 | 5′-GGCGGCGAGCCTCCCTCGCGCCATCAG-3′ |

Primers for Solexa libraries:

| | | |
|---|---|---|
| Forward: | SEQ ID NO: 7 | 5′-ACACTCTTTCCCTACACGA-3′ |
| Reverse: | SEQ ID NO: 8 | 5′-CAAGCAGAAGACGGCATA-3′ |
| UTBP-3: | SEQ ID NO: 9 | 5′-GGCGGCGAACACTCTTTCCCTACACGA-3′ |

Universal Probe Sequence:

| | | |
|---|---|---|
| UPL#149 | SEQ ID NO: 10 | 5′-CCGCCGCT-3′ |

The primers were chosen to be used with particular adapters supplied by a commercial manufacturer, after the library was created according to the protocol for the particular sequencing methodology to be used. Blunt end ligation and several rounds of PCR amplification were used to attach the adapters. Other methods of attachment of adapters to the sequence of interest are known and may be employed, for example Fast-Link from Epicentre Biotechnologies. Other primers will be apparent given the present disclosure, and will be chosen to permit amplification based on hybridization to adapters as used in the library preparation protocol.

### REFERENCES

1. Mackelprang, R., Rubin, E. M. (2008). PALEONTOLOGY: New Tricks with Old Bones. Science, 321(5886), 211-212.
2. David A. C. Simpson, Susan Feeney, Cliona Boyle, Alan W. Stitt. (2000) Retinal VEGF mRNA measured by SYBR Green I fluorescence: A versatile approach to quantitative PCR. Molecular Vision 2000; 6:178-183
3. Jones L J, Yue S T, Cheung C Y, Singer V L. RNA quantitation by fluorescence-based solution assay: RiboGreen reagent characterization. Anal. Biochem. (1998) 265:368-374.
4. Margulies M, et al. (2005) Genome sequencing in microfabricated high-density picoliter reactors. Nature 437:376-380.
5. Meyer M, et al (2008) From micrograms to picograms: quantitative PCR reduces the material demands of high-throughput sequencing. Nucleic Acids Research 36: (1) e5
6. Ricicova M, Palkova Z. Comparative analyses of Saccharomyces cerevisiae RNAs using Agilent RNA 6000 Nano Assay and agarose gel electrophoresis. FEMS Yeast Res. (2003) 4:119-122
7. Warren, L.; Bryder, D.; Weissman, I. L.; Quake, S. R. Proc. Natl. Acad. Sci. U.S.A. 2006, 103 (47), 17807-12.
8. Ottesen, E. A.; Hong, J. W.; Quake, S. R.; Leadbetter, J. R. Science 2006, 314 (5804), 1464-7.
9. Blow, M et al., Identification of ancient remains through genomic sequencing Genome Res. 18:1347-1353, 2008.

### CONCLUSION

The above specific description is meant to exemplify and illustrate the invention and should not be seen as limiting the scope of the invention, which is defined by the literal and equivalent scope of the appended claims. Numerous modifications to the exemplified methods and materials will be apparent, given the present teachings. For example, the present digital PCR methods may be used with RNA as well as DNA. In this case, cDNA copies are made and then amplified by DNA polymerase-based PCR. Different primers may be used for cDNA synthesis. Specific templates, based on genetic sequences in the chromosomes of interest are preferred. See, Bustina et al., “Pitfalls of Quantitative Real-Time Reverse-Transcription Polymerase Chain Reaction,” Journal of Biomolecular Techniques, 15:155-166 (2004). It may also be possible to design primers and probes to other UTs (adapters), where there is not specifically a 5′ and 3′ adapter. For example, primers may be designed which themselves give a signal upon binding and amplification. For example, Scorpion® primer/probes, available from Sigma Aldrich, may be used. In Scorpion primers, the probe is physically coupled to the primer which means that the reaction leading to signal generation is a unimolecular one. This is in contrast to the bi-molecular collisions required by other technologies such as TaqMan® or Molecular Beacons. Also, dyes may be used in place of the exemplified UT probe to detect the amplified product. Also, Lux^{tm} fluorogenic primers, as currently marketed by Invitrogen may be used. The Lux primer pairs include one fluorogenicly labeled primer. When the primer is extended, it becomes fluorogenic. As another alternative, the 5′ adapter and 3′ adapter may in certain embodiments, not be completely physically at the 5′ and 3′ ends of the nucleic acid molecule to be sequenced.

Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference, as needed for the purpose of further describing and enabling the method or material referred to, as provided for in 37 CFR 1.57 or succeeding patent rules.

What is claimed is:

1. A method for determining concentration of DNA molecules in a DNA sequencing library, comprising:
(a) providing a library comprising a plurality of individual DNA molecules, said molecules individually having attached thereto a 5′ adapter and a 3′ adapter, said 5′ adapter and 3′ adapter spanning a sequence of interest;
(b) distributing said individual DNA molecules from the library to a number of individual reaction areas, wherein the percentage of reaction areas containing one or more of the DNA molecules is greater than 0 percent and less than 100 percent;
(c) amplifying DNA molecules, if present in a reaction area, using a forward primer binding to the 5′ adapter and a reverse primer binding to the 3′ adapter; and
(d) generating a signal in each reaction area containing amplified molecules, whereby
the number of reaction areas generating a signal is indicative of the quantity of DNA molecules in the sample.

2. The method of claim 1 where the step of generating a signal comprises generating a fluorescent signal.

3. The method of claim 1 wherein said step of distributing is done in a microfluidic device adapted for carrying out PCR reactions in individual reaction areas.

4. The method of claim 1 wherein said step of distributing is done by either a microfluidic device, a gel, an emulsion, a bead, or a multiwell plate.

5. The method of claim 1 where the forward primer contains a complementary sequence for binding of a probe used for said generating of a signal.

6. The method of claim 4 further comprising the step of adding forward primer both with and without the complementary sequence during the amplification reaction.

7. The method of claim 1 where the amplification is done in at least 100 reaction areas.

8. The method of claim 1 wherein generating a signal is done with a molecule that contains a fluorescent molecule and a quencher which are separated during said amplifying to generate fluorescence.

9. The method of claim 8 where the probe binds to a primer and contains from 7 to 12 bases which are complementary to the primer binding site and a fluorescent dye and quencher at opposite ends.

10. The method of claim 9 where the probe contains at least one normatural base to increase binding affinity.

11. A method for sequencing DNA where the sequencing process begins with a library of DNA molecules, comprising:
(a) obtaining a sample of individual DNA molecules from the library to be sequenced;
(b) attaching a 5′ adapter on a 5′ end of each molecule and a 3′ adapter on a 3′ end of each molecule, each 5′ adapter and 3′ adapter having the same sequence;
(c) distributing said individual molecules to a number of individual reaction areas, each reaction area having on average no more than about one to two molecules per area;
(d) amplifying a single molecule, if present in a reaction area, using a forward primer binding to the 5′ adapter and a reverse primer binding to the 3′ adapter on the single molecule;
(e) generating a signal by means of a probe which binds to a sequence defined on a forward primer or a reverse primer, whereby
the number of reaction areas generating a signal is indicative of the quantity of DNA molecules in the sample; and
(f) sequencing the sample using an amount of DNA determined by the quantity of DNA as determined in step (e).

12. A method of quantifying nucleic acid molecules in a sample, each of said nucleic acid molecules having a 5′ region and a 3′ region, each 5′ region of identical, known sequence, and each 3′ region being of identical, known sequence, comprising:
(a) distributing said individual nucleic acid molecules to a number of individual reaction areas, each reaction area having a calculated average number of nucleic acid molecules per area;
(b) amplifying a single nucleic acid molecule, if present in a reaction area, using a forward primer binding to the 5′ region and a reverse primer binding to the 3′ region on the single molecule; and
(e) generating a signal which is dependent upon amplification, whereby the number of individual reaction areas generating a signal is indicative of the quantity of the nucleic acid molecules in the sample.

13. The method of claim 12 where the distributing is done in a microfluidic device.

14. The method of claim 12 where the identical known sequences are adapter molecules comprising identical, known sequences attached to the nucleic acid molecules in the sample and used for sequencing.

15. The method of claim 12 where the generating a signal comes from a probe which hybridizes to a primer used in said amplifying.

16. The method of claim 15 where the step of amplifying comprises amplifying with a primer containing a probe binding region and a competing primer not containing a probe binding region.

17. A method for using a universal template for a probe, said probe being fluorescent, said method comprising a real time PCR reaction, said method being characterized by the use as said probe of a probe having a length of between 8 and 12 bases, at least one of said bases being a normatural base for higher binding to the template.

18. A hydrolysis probe having a sequence complementary to a portion of a PCR primer, said portion of the PCR primer being non-complementary to a template binding sequence in the primer but complementary to the probe.

19. A kit comprising a hydrolysis probe having a sequence complementary to a portion of a PCR primer, said portion of the PCR primer being non-complementary to a template binding sequence in the primer but complementary to the probe, and a primer binding to the probe.

20. The kit of claim 19 where the primer binds to an adapter molecule attached to a DNA molecule to be sequenced.

21. The kit of claim 20 further comprising a pair of primers, each primer binding, respectively to a 5′ adapter and a 3′ adapter.

22. A kit for quantifying a population of nucleic acid strands, comprising:
(a) 5′ adapters and 3′ adapters for the nucleic acid strands, each 5′ adapter and 3′ adapter having the same sequence;
(b) forward and reverse primers complementary to the 5′ and 3′ adapters, respectively, said forward primer having a non-complementary region for providing a sequence for binding of a labeled probe; and
(c) said labeled probe having a fluorescer-quencher pair which provides an optical signal during amplification, said labeled probe further characterized as having between 7 and 15 bases, and having a non-natural base for increasing binding.
